(19) United States
(12) Patent Application Publication
(10) Pub. No.: US 2006/0120981 A1
(43) Pub. Date: Jun. 8, 2006

(54) Apparatus and process for the production of multicoloured poured cosmetic products with various shapes

(75) Inventor: Nadia Avalle, Vallese (CH)

Correspondence Address: AKERMAN SENTERFITT, P.O. BOX 3188, WEST PALM BEACH, FL, 33402-3188, US

(73) Assignee: Intercos S.p.A., Milano (IT)

(21) Appl. No.: 11/291,581

(22) Filed: Dec. 1, 2005

(30) Foreign Application Priority Data
Dec. 3, 2004 (IT) MI2004A002331

(51) Int. Cl. A61K 8/00; B28B 1/54

(52) U.S. Cl. 424/63; 425/6

(57) ABSTRACT

An apparatus and a process for the production of multicolour poured cosmetic products with various shapes are described. Said apparatus comprises a melter (8) for the preparation of a melt cosmetic composition (9) thrust by pumping means (10) into a dose meter (12) controlled by a control unit (13) for the controlled delivery of a dose of cosmetic composition (9) into a container (31). Said dose meter (12) and said container (31) are mobile with respect to each other.

The present invention concerns a unit and a process for the production of multicolour poured cosmetic products with various shapes.

By poured cosmetic product an anhydrous cosmetic product is meant that is made up of a formula comprising raw materials of fat and oleos nature, colouring agents and other, which, in order to be able to be transformed into a usable product, must be subject to a melting process.

During the melting stage, the product becomes fluid and the fluid thus obtained can be dosed inside a cavity, of which it will then take the shape, by filling it up.

Once let cool down, the cosmetic product will be hardened so that it can therefore be easily removed for its use.

Poured products are, for example, the reds for lips, either poured or in stick, and the poured glosses.

The techniques currently in use to produce poured cosmetic products comprise rather simple pouring systems, generally made up of a melter provided with an agitator and a dose metering unit which can be a gear pump or a volumetric pneumatic dose meter right downstream of the same melter.

The initial product is introduced into the melter, brought to the melting temperature and then hot dosed inside the container.

Such container can be made up of a metal tray, commonly called bottom or godet, or directly of the final container (or “packaging”).

The majority of poured cosmetic products on the market is made up of single-colour products, placed in a single container.

In order to give the possibility to have several colours available at the same time, usually the various colours get poured each one into single bottoms and then these are inserted in appropriate housings, single or multiple, inside the final packaging, by placing the different colours next to each other.

There are also known systems to obtain different colours and shapes, joined together within the same bottom.

Such systems provide the use of metallic diaphragms in order to create the single shapes and then the joining of these with a poured cosmetic product which acts as a union.

The procedure to be followed is the following:

a) inside the bottom or directly in the packaging a metallic insertion with the shape of one of the shapes to be provided is placed;

b) one of the products gets poured into the part of housing that is left free, around the metallic insert;

c) the first poured product is cooled down and, once hardened, the metallic insert gets extracted;

d) the second product gets poured into the empty cavity which was created by extracting the insert.

With this system it is possible to pour two or three shapes different from each other, and/or different colours, inside the same container.

Unfortunately this technology has still today many limits, for example:

a) possibility of adhesion of the poured product to the metal insert;

b) excessive limits in the production of the metallic shapes;

c) limitations in the width of the provided shapes;

d) difficulty in sliding of the poured product in narrow shapes and in shapes with acute angles;

e) excessive number of operating steps;

f) large volumes of the equipments for the inserts.

A first object of the present invention is to provide an apparatus which allows to provide cosmetic products with shapes and colours very different from each other without the above mentioned problems.

A second object of the present invention is to provide a process which allows to provide cosmetic products with shapes and colours very different from each other without the above mentioned problems.

According to the invention said first object is provided by an apparatus for the production of multicolour poured cosmetic products with various shapes, comprising a melter for the preparation of a melt cosmetic composition pushed by pumping means into a dose meter controlled by a control unit for the controlled delivery of a dose of cosmetic composition into a container, characterised in that said dose meter and said container are mobile with respect to each other.

According to the invention said second object is attained with a process for the production of multicolour poured cosmetic products with various shapes, comprising the steps of:

preparation of a melted pouring cosmetic composition inside a melter (8),

pumping of the cosmetic composition towards a dose meter,

dosage of the cosmetic composition into a container,

characterised in that said dose meter and said container are movable with respect to each other.

Shapes of colour or composition different from each other can be obtained directly by dosing different cosmetic compositions inside the same container without the use of diaphragms separators.

The characteristics of the present invention will be made evident by the following detailed description of an embodiment thereof which is illustrated as a non-limiting example in the enclosed drawings, in which:

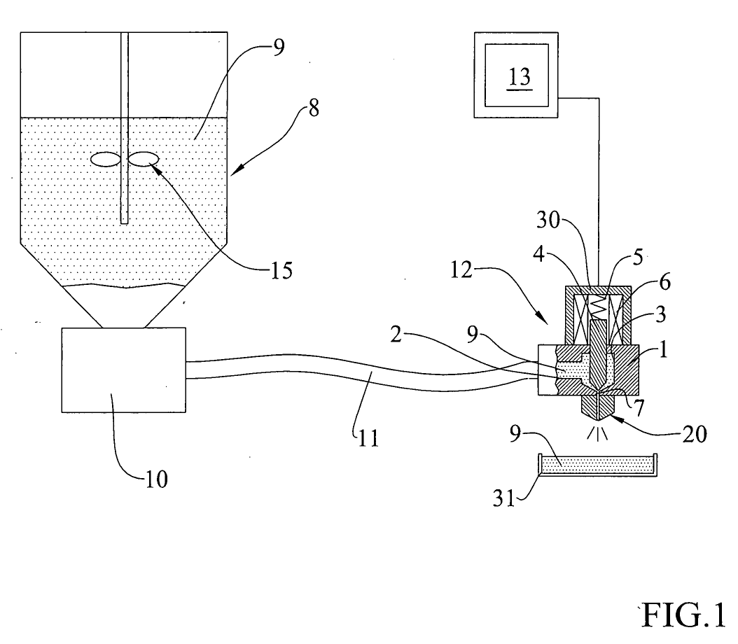

FIG. 1 shows a side view of an apparatus according to the present invention;

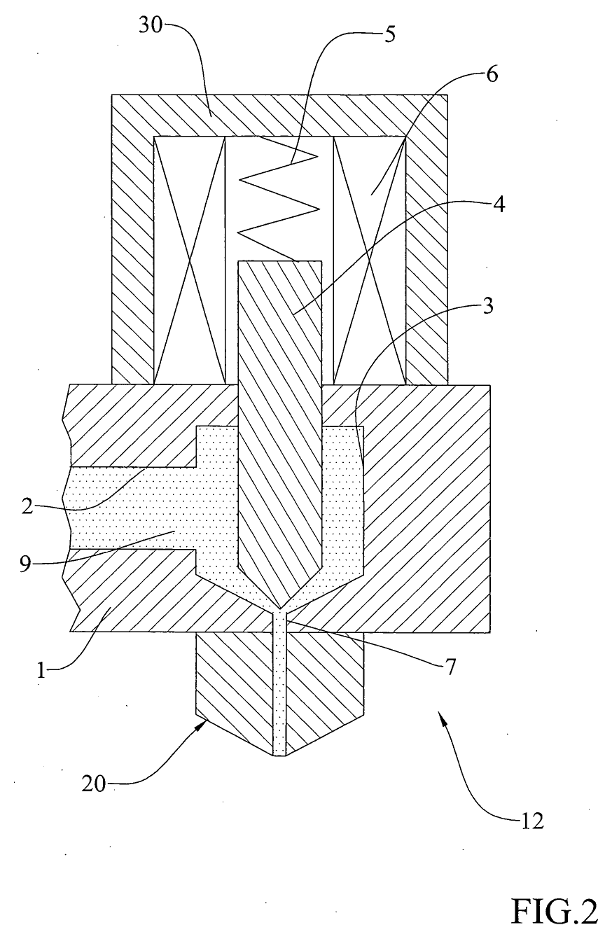

FIG. 2 shows a magnified section view of the dose meter according to the present invention;

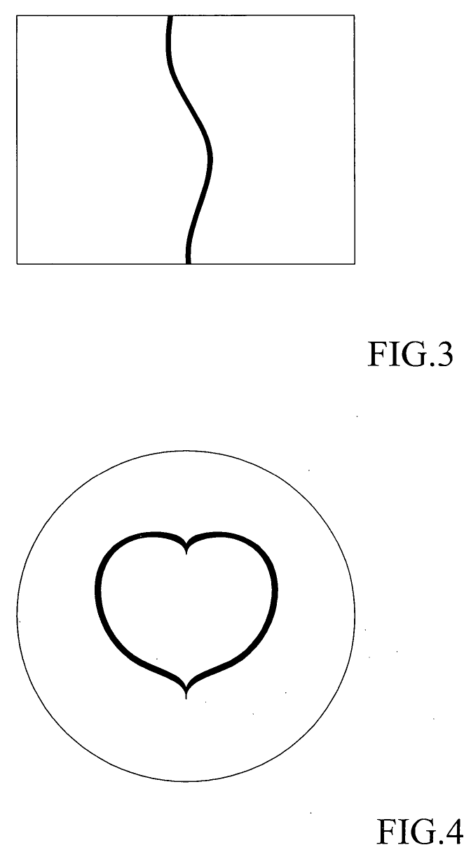

FIGS. 3 and 4 show possible shapes of cosmetic product with several colours obtainable with the apparatus according to the invention.

The apparatus of FIG. 1 comprises a melter 8 for the preparation of a melt cosmetic composition 9 thrust by a pump 10, through a heated flexible connection hose 11, into a mobile dose meter 12.

The melter 8 comprises internally a screw agitation device 15.

The mobile dose meter 12 comprises a mobile support 30 to which a dose metering head 1 is fastened within which an inlet channel 2 and an outlet hole 7 are obtained, downstream of which a delivery nozzle 20 is placed, which communicate with each other by means of an intermediate chamber 3 within which a plug 4 is housed that is supported by a compression spring 5, axially mobile and controlled by an excitable electric coil 6 controlled by a remote control unit 13, which also controls the horizontal and vertical movement of the dose meter 12.

The nozzle 20 delivers the composition 9 into a container 31 arranged below.

In operation, the melter 8 gets filled with the cosmetic composition 9 involved, that is taken to the desired pouring temperature. The screw agitation device 15 allows to maintain the cosmetic composition 9 homogenous.

Downstream of the melter 8 a gear pump 10 is placed, which allows to push the composition 9 outside through the outlet hole of the same pump 10.

Through the heated flexible hose 11, the fluid cosmetic composition 9, maintained at the desired temperature and under pressure, reaches the mobile dose meter 12 placed at a distance (even several meters) from the same melter 8.

The dose meter 12 is mobile in all directions and it is capable, in virtue of the control unit 13, to deliver the cosmetic composition 9 in the desired amounts and for the desired time, by making it pass through the delivery nozzle 20.

By varying the dimension of the nozzle 20, the delivery time and the pressure of the pump 10, it is possible to vary in consistent way the amount of cosmetic composition 9 being delivered.

The cosmetic composition 9 enters the dosing head 1 through the inlet channel 2 and it reaches the chamber 6.

In order to exit, the composition 9 must be able to pass through the outlet hole 7 closed by the plug 4 which, by raising and lowering themselves even for very short times under the control of the control unit 13, allows the dosage, even in very small amounts, of the cosmetic composition 9 (FIG. 2).

The dosing head 1 is fastened onto the mobile support 30 managed by the control unit 13 which allows the dosage of the composition both at various heights, and on all the surface, thus writing and designing various forms and shapes different from each other.

It is possible, for example, to create very thin shapes, similar to small walls of product, with thickness even smaller than a millimetre, which create sectors inside the container 31.

Such walls can be rectilinear, undulated, shaped in many different ways, up to reproducing a wide variety of different shapes.

The cavities which have thus been formed get subsequently filled with other melt product, of other colour, so as to create a polychrome figure.

Naturally it is possible also to invert the dosage process, that is to cause movement, still managed by a control unit 13, of the bottom part, where the container 31 is housed.

1. Apparatus for the production of multicolour poured cosmetic products with various shapes, comprising a melter (8) for the preparation of a melt cosmetic composition (9) thrust by pumping means (10) into a dose meter (12) controlled by a control unit (13) for the controlled delivery of a dose of cosmetic composition (9) into a container (31), characterised in that said dose meter (12) and said container (31) are mobile with respect to each other.

2. Apparatus according to claim 1, characterised in that said dose meter (12) comprises a mobile support (30) controlled by said remote control unit (13), a dosing head (1) being fastened to said mobile support (30) within which an inlet channel (2) and an outlet hole (7) are obtained, downstream of which a delivery nozzle (20) is placed, which communicate with each other by means of an intermediate chamber (3) inside which a plug (4) is housed that is biased by a spring (5), which is axially mobile and controlled by an excitable coil (6) controlled by said remote control unit (13).

3. Apparatus according to claim 1, characterised in that said pumping means consist of a gear pump (10).

4. Apparatus according to claim 1, characterised in that said pumping means (10) and said dose meter (12) are connected by a flexible hose (11).

5. Apparatus according to claim 1, characterised in that said flexible hose (11) is heated.

6. Apparatus according to claim 1, characterised in that said melter (8) is heated and is provided with an agitation device (15) in order to maintain the cosmetic composition (9) homogenous.

7. Process for the production of multicolour poured cosmetic products with various shapes, comprising the steps of:
preparation of a melt cosmetic composition (9) for pouring into a melter (8),
pumping of the cosmetic composition (9) towards a dose meter (12),
dosage of the cosmetic composition (9) into a container (31), characterised in that said dose meter (12) and said container (31) are movable with respect to each other.
